(19) United States
(12) Patent Application Publication
(10) Pub. No.: US 2025/0059680 A1
(43) Pub. Date: Feb. 20, 2025

(54) NEUTRON-GENERATING TARGET AND METHOD FOR PRODUCING THE SAME

(71) Applicant: NGK INSULATORS, LTD., Nagoya-City (JP)

(72) Inventors: Satoshi OTA, Nagoya-City (JP); Masato SAKAKIBARA, Handa-City (JP)

(73) Assignee: NGK INSULATORS, LTD., Nagoya-City (JP)

(21) Appl. No.: 18/731,546

(22) Filed: Jun. 3, 2024

(30) Foreign Application Priority Data
Aug. 15, 2023 (JP) 2023-132339

(51) Int. Cl. C30B 29/60; A61N 5/10; C30B 23/02; C30B 23/06; C30B 29/02

(52) U.S. Cl. CPC C30B 29/605; A61N 5/10; C30B 23/025; C30B 23/066; C30B 29/02; A61N 2005/109

(57) ABSTRACT

There is provided a neutron-generating target, including a beryllium layer composed of a plurality of beryllium crystal grains having a columnar structure that has grown in a thickness direction, wherein the beryllium layer has gaps between the plurality of beryllium crystal grains.

### CROSS-REFERENCE TO RELATED APPLICATION

This application claims priority to Japanese Patent Application No. 2023-132339 filed Aug. 15, 2023, the entire contents of which are incorporated herein by reference.

### BACKGROUND OF THE INVENTION

### 1. Field of the Invention

The present invention relates to a neutron-generating target and a method for producing the same.

### 2. Description of the Related Art

Neutrons are used for applications such as the boron neutron capture therapy (BNCT) that is one of cancer therapies, the neutron diffraction that analyzes and identifies the structure of a substance, or the neutron imaging that is one kind of nondestructive inspections. As a source of neutrons, an accelerator, which generates neutrons by, for example, irradiating a metal target with accelerated protons, has been known, in addition to conventionally used nuclear reactors. As the accelerator, there are large accelerators having a size of several kilometers, which are used in facilities such as J-PARC and SPring-8. However, in terms of installation easiness, small accelerators (e.g., with an installation area of a device main body less than 50 m²) are more favorable.

The small accelerator generates smaller accelerated energy of protons than the large accelerator. Therefore, a target that generates neutrons even with small energy is needed. In this point, beryllium has been known as a material of the target for generating neutrons used in, for example, the small accelerator. In the beryllium target, bubbles (swelling) called blistering are generated during use. A problem is that when the bubbles become large, they are finally ruptured. That is, the diffusion rate of hydrogen is very low in beryllium, and accelerated and emitted protons stagnate in beryllium to become hydrogen gas. Then, the thus-generated hydrogen gas appears as blistering.

Several techniques in order to prevent generation of blistering are proposed. For example, Non-Patent Literature 1 (T. Rinckel et al., “Target Performance at the Low Energy Neutron Source” Physics Procedia 26, pp. 168-177 (2012)) proposes that protons are allowed to pass without stagnating in a target by, for example, increasing accelerated energy of protons or decreasing the thickness of the target, to stop protons on the back surface of the target in cooling water. Patent Literature 1 (JP6713653B) discloses that, with regard to a neutron-generating target including a neutron-generating target material containing beryllium and a blistering-resistant intermediate material made of a metal or the like capable of accumulating hydrogen, the thickness of the neutron-generating material is equal to or lower than the range of the incident neutron. According to the neutron-generating target, it is believed that stopping the emitted protons stops in the blistering-resistant intermediate material prevents protons from being hydrogenated inside the neutron-generating target material and prevents generation of blistering.

### CITATION LIST

### Patent Literature

Patent Literature 1: JP6713653B

### Non-Patent Literature

Non-Patent Literature 1: T. Rinckel et al., “Target Performance at the Low Energy Neutron Source”, Physics Procedia 26, pp. 168-177 (2012)

### SUMMARY OF THE INVENTION

However, a problem is that when the accelerated energy of protons becomes higher, an accelerator, a radiation shielding body and the like need to be enlarged. In addition, when the thickness of a target is thin, a decrease in the amount of generated neutrons, a decrease in mechanical strength of the structure, and the like may occur. Therefore, regardless of the accelerated energy of protons and the thickness of the target, a neutron-generating target capable of preventing generation of blistering is desired.

The present inventors recently found that, in a beryllium layer composed of a plurality of beryllium crystal grains, it is possible to provide a neutron-generating target capable of preventing generation of blistering, by providing gaps between the beryllium crystal grains.

Therefore, an object of the present invention is to provide a neutron-generating target capable of preventing generation of blistering.

The present invention provides the following aspects:

### [Aspect 1]

A neutron-generating target, comprising a beryllium layer composed of a plurality of beryllium crystal grains having a columnar structure that has grown in a thickness direction, wherein the beryllium layer has gaps between the plurality of beryllium crystal grains.

### [Aspect 2]

The neutron-generating target according to aspect 1, wherein the plurality of beryllium crystal grains have a frost column-like structure as a whole.

### [Aspect 3]

The neutron-generating target according to aspect 1 or 2, wherein a distance between centers of the plurality of beryllium crystal grains adjacent to each other is from 2 to 50 μm.

### [Aspect 4]

The neutron-generating target according to any one of aspects 1 to 3, wherein the beryllium crystal grains have an aspect ratio of from 1 to 3,000.

### [Aspect 5]

The neutron-generating target according to any one of aspects 1 to 4, wherein the beryllium layer has helium gas permeability.

### [Aspect 6]

The neutron-generating target according to any one of aspects 1 to 5, wherein the beryllium layer is a physical vapor deposition film.

### [Aspect 7]

The neutron-generating target according to any one of aspects 1 to 6, wherein the beryllium layer has a thickness of from 0.02 to 6 mm.

### [Aspect 8]

The neutron-generating target according to any one of aspects 1 to 7, further comprising a substrate on one surface of the beryllium layer.

### [Aspect 9]

The neutron-generating target according to aspect 8, wherein the substrate is composed of copper.

### [Aspect 10]

A method for producing a neutron-generating target, the method comprising allowing a plurality of beryllium crystal grains to grow on one surface of a substrate in a thickness direction of the substrate by a physical vapor deposition, thereby obtaining a beryllium layer having gaps between the plurality of beryllium crystal grains.

### [Aspect 11]

The method for producing a neutron-generating target according to aspect 10, wherein the physical vapor deposition is at least one selected from the group consisting of a resistance heating evaporation method, an electron-beam evaporation method, and a sputtering method.

### BRIEF DESCRIPTION OF THE DRAWINGS

FIG. 1 is a schematic cross-sectional view of one aspect of a neutron-generating target of the present invention.

FIG. 2 is a graph plotting a leak rate relative to elapsed time in a helium leak testing using a beryllium target of Example 1.

FIG. 3 is a graph plotting a leak rate relative to elapsed time in a helium leak testing using a beryllium target of Example 2.

FIG. 4 is an SEM image (magnification of 1,000 folds) obtained by observing the beryllium target of Example 1 from just above.

FIG. 5 is an SEM image (magnification of 3,000 folds) obtained by observing the beryllium target of Example 1 from just above.

FIG. 6 is an SEM image (magnification of 500 folds) obtained by observing a polished surface of the beryllium target of Example 1.

FIG. 7 is an SEM image (magnification of 2,000 folds) obtained by observing a polished surface of the beryllium target of Example 1.

FIG. 8 is an optical microscope image (magnification of 200 folds) by observing a cross section of the beryllium target of Example 2 after etching.

FIG. 9 is an optical microscope image (magnification of 500 folds) by observing a cross section of the beryllium target of Example 2 after etching.

### DETAILED DESCRIPTION OF THE INVENTION

### Neutron-Generating Target

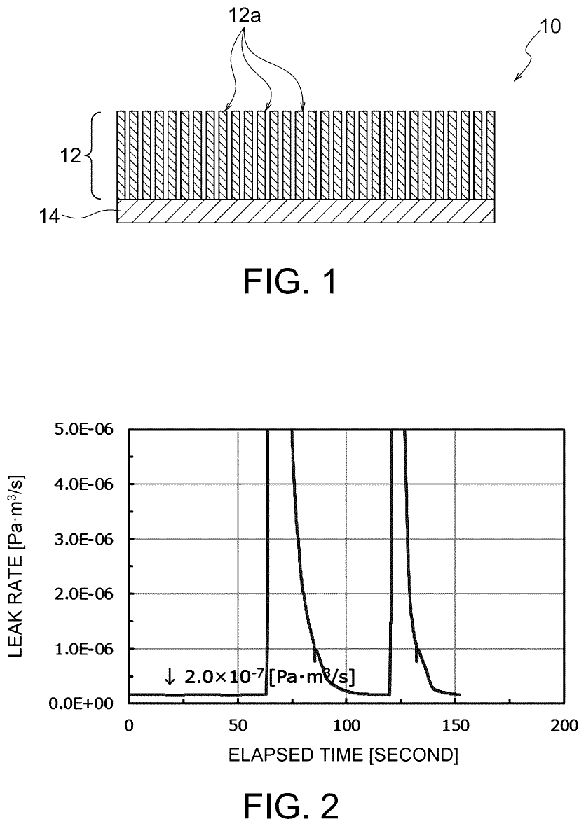

As schematically shown in FIG. 1, a neutron-generating target 10 of the present invention includes a beryllium layer 12. Optionally, the neutron-generating target 10 may further include a substrate 14 on one surface of the beryllium layer 12. The beryllium layer 12 is composed of a plurality of beryllium crystal grains 12a. Each of the beryllium crystal grains 12a has a columnar structure that has grown in a thickness direction of the beryllium layer 12. The beryllium layer 12 includes gaps between the plurality of beryllium crystal grains 12a. As described above, in the beryllium layer 12 composed of the plurality of beryllium crystal grains 12a, when the gaps are provided between the beryllium crystal grains 12a, it is possible to provide the neutron-generating target 10 capable of preventing generation of blistering.

A conventional beryllium target is typically composed of a block material (sintered body) of beryllium. Therefore, there is no gap between beryllium crystal grains. As described above, it is believed that accelerated and emitted protons stagnate in the beryllium layer to turn into hydrogen gas, thus generating blistering. On the other hand, in the neutron-generating target 10 of the present invention, even when the accelerated and emitted protons turn into hydrogen gas in the beryllium layer 12, the hydrogen gas passes through voids between the beryllium crystal grains 12a to be released to the outside. Therefore, regardless of accelerated energy of protons or the thickness of the beryllium layer 12, generation of blistering can be prevented.

Each of the plurality of beryllium crystal grains 12a constituting the beryllium layer 12 is preferably composed of a simple substance of the beryllium metal. As long as a desired amount of neutrons is generated, the beryllium layer 12 may include, for example, a beryllium compound or beryllium alloy other than the simple substance of the beryllium metal. In addition, the beryllium layer 12 may include inevitable impurities contributed from, for example, the material component or the film forming process.

As shown in FIG. 1, each of the beryllium crystal grains 12a has a columnar structure that grows in a thickness direction of the beryllium layer 12. According to a preferable embodiment of the present invention, the plurality of beryllium crystal grains 12a has a frost column-like structure as a whole. In such a structure, it is said that the beryllium layer 12 has three-dimensional gaps between crystal grains, and the hydrogen gas generated inside the beryllium layer 12 is more efficiently released. As a result, generation of blistering is more effectively prevented. In the schematic view shown in FIG. 1, each of the beryllium crystal grains 12a is drawn as if uniformly extended from the lower surface to the upper surface of the beryllium layer 12. However, the beryllium crystal grains 12a are not limited to such a construction and may include grain boundaries between the lower surface and the upper surface of the beryllium layer 12.

A distance between centers of the plurality of beryllium crystal grains 12a adjacent to each other is preferably from 2 to 50 μm and more preferably from 2 to 25 μm. Within such a range, hydrogen gas is more efficiently released, and thus generation of blistering is more effectively prevented. The distance between centers of crystal grains can be measured by observing the cross section of the beryllium layer 12 with a scanning electron microscope.

The beryllium crystal grains 12a preferably has an aspect ratio of 1 or more, more preferably 2 or more, and still more preferably 5 or more. Within such a range, the hydrogen gas generated in the beryllium layer 12 easily move toward the thickness direction of the beryllium layer 12. As a result, generation of blistering is more effectively prevented. The upper limit of the aspect ratio of the beryllium crystal grains 12a is not particularly limited, but is typically 200 or less. In the present invention, the aspect ratio of the beryllium crystal grains 12a means a ratio (=L_{T}/L_{w}) of the maximum length L_{T} of the beryllium crystal grains 12a in a thickness direction of the beryllium layer 12 to the maximum length L_{w} of the beryllium crystal grains 12a in a width direction of the beryllium layer 12. The maximum length L_{w} of the beryllium crystal grains 12a in a width direction of the beryllium layer 12 is not particularly limited, but is typically from 2 to 50 μm. The maximum length L_{T} of the beryllium crystal grains 12a in a thickness direction of the beryllium layer 12 is not particularly limited, but is typically 2 μm or more. The maximum length L_{w}, the maximum length L_{T}, and the aspect ratio can be measured by observing the cross section of the beryllium layer 12 with a scanning electron microscope.

The beryllium layer 12 preferably has helium gas permeability. Here, a dynamic molecular size of helium is 0.26 nm, which is slightly smaller than a molecular size of hydrogen of 0.289 nm. Therefore, when the beryllium layer 12 has a helium gas permeability, it can be said that hydrogen gas is also further released from the beryllium layer 12. The “having helium gas permeability” in the present invention means that when the helium leak testing is performed according to a vacuum spray method (spray method) stipulated in Annex 1 of JIS Z2331:2006, leak of helium gas is detected. In the helium leak testing, the maximum value of the leak rate (Pa·m³/s) detected with a leak detector after helium gas is sprayed is preferably 10 times or larger (preferably 100 times or larger) than the leak rate detected with a leak detector before helium gas is sprayed.

The beryllium layer 12 preferably has a thickness of from 0.02 to 6 mm. Within such a range, a desired amount of neutrons is easily generated, and a sufficient strength of the target can be ensured. A surface area (i.e., a surface area of a surface to which protons are irradiated) when the beryllium layer 12 is viewed in a plane manner can be appropriately determined depending on the specification of a neutron generator to be used (e.g., a small accelerator). The surface area is not particularly limited, but is typically from 30 to 400 cm².

The beryllium layer 12 may be prepared by any method but is preferably a physical vapor deposition film. A preferable production method of the neutron-generating target 10 including the beryllium layer 12 will be described later.

As described above with reference to FIG. 1, the neutron-generating target 10 may include the substrate 14 on one surface of the beryllium layer 12. The substrate 14 can function as a member configured to cool the beryllium layer 12 to which accelerated protons are irradiated.

The substrate 14 is preferably composed of copper. This makes it possible to efficiently cool the beryllium layer 12. The thickness of the substrate 14 is not particularly limited, but is typically 0.1 mm or more.

### Production Method of Neutron-Generating Target

According to a preferable embodiment of the present invention, a production method of the neutron-generating target 10 is provided. This method includes growing the plurality of beryllium crystal grains 12a on one surface of the substrate 14 by the physical vapor deposition in a thickness direction of the substrate 14. This makes it possible to obtain the beryllium layer 12 having gaps between the plurality of beryllium crystal grains 12a.

Preferable examples of the physical vapor deposition include a resistance heating evaporation method, an electron-beam evaporation method, and a sputtering method. More preferably, the resistance heating evaporation method and the electron-beam evaporation method, which are excellent in economical efficiency, are exemplified. Regarding the film formation with the resistance heating evaporation method, the deposition is preferably performed by heating beryllium as a raw material to 1287° C. (melting point of beryllium) or higher under conditions of degree of vacuum of 2×10⁻² Pa or less and a substrate temperature of from 350 to 550° C. until the beryllium layer has a desired thickness. Regarding the film formation with the electron-beam evaporation method, the same conditions with those of the resistance heating evaporation method may be used except that voltage and electric current are controlled to melt beryllium. Regarding the film formation with the sputtering method, after a high vacuum is created inside a chamber, the vacuum is filled with Ar gas of about 1 Pa, and voltage may be applied between a substrate and beryllium until a desired thickness of a beryllium layer is achieved.

Beryllium as a raw material used for the physical vapor deposition is preferably obtained by processing beryllium ingot produced by vacuum casting to small pieces. The purity of the beryllium ingot is preferably 98% by weight or more, and more preferably 99% by weight or more.

### EXAMPLES

The present invention will be described in more detail by the following Examples.

### Example 1

As a neutron-generating target of the present invention, a beryllium target was prepared by the following procedure.

### (1) Providing Beryllium Ingot

Beryllium ingot (purity: 99% by weight) obtained by vacuum casting was provided. The beryllium ingot was produced by the following manner. Particulate metallic beryllium was charged into a magnesium oxide crucible in a vacuum melting furnace, and the inside of the furnace was maintained at about 1300° C. until the beryllium was melted. After beryllium was completely melted, the temperature and the degree of vacuum of the furnace were increased to remove impurities. As described above, after refinement in the vacuum melting furnace was completed, the melted product was poured into a graphite crucible in vacuum, and was solidified, to make beryllium ingot.

### (2) Preparing Target by Physical Vapor Deposition

Provided beryllium ingot was cut into small pieces, and the small pieces were set in a crucible in a vacuum chamber. On one surface of a commercially available product of a pure copper plate (thickness of 0.15 mm) provided as a deposition substrate, a beryllium layer having a thickness of 0.12 mm was formed by the resistance heating evaporation method using the following device and conditions.
Device: resistance heating evaporation equipment (available from Sanyu Electron Co., Ltd, product number: SVC-700TMSG/7PS80)
Beryllium heating temperature: 1300° C.
Deposition time: 60 minutes
Degree of vacuum: 2×10⁻² Pa or less
Substrate temperature: 450° C.

For various evaluations that will be described later, the pure copper plate on which the beryllium layer had been formed was subjected to an etching removal with nitric acid to obtain a beryllium target having a diameter of 50 mm and a thickness of 0.12 mm.

### Example 2 (Comparison)

A beryllium target was prepared by a conventional powder sintering method. The specific procedure is as follows.

### (1) Providing Beryllium Ingot

The same beryllium ingot as that of Example 1 was provided.

### (2) Preparing Target by Powder Sintering

The provided beryllium ingot was mechanically crushed, and then resulting crushed product was sieved to obtain beryllium powder having a granularity of 300 meshes. The beryllium powder was sintered by a vacuum hot press method, to form a molded body. Specifically, the beryllium powder was filled into a mold in the vacuum furnace, and a pressure of 8 MPa was applied in the vertical direction while it was heated under vacuum within a temperature range of from 1025 to 1125° C., to obtain a molded body. This molded body was cut so as to have a thickness of 10 mm, and hot rolling and soft annealing were repeated. At this time, the temperature ranges of the hot rolling and the soft annealing were from 750 to 950° C. As described above, a beryllium target having a diameter of 50 mm and a thickness of 0.025 mm was obtained.

### Various Evaluations

The beryllium targets obtained in Examples 1 and 2 were subjected to various evaluations as shown below.

### <Helium Leak Testing>

The helium leak testing was performed on the beryllium target according to the vacuum spray method (spray method) of JIS Z2331:2006. Specifically, the beryllium target was fixed in a sample holder inside a work connected to a leak detector (available from ULVAC, Inc., HELIOT901W1). After vacuum was created inside of the work, helium gas was sprayed from the outside of the work using a spray probe, and the helium gas leaked into the work was detected by the leak detector. The helium gas was sprayed two times in each Example.

As a result of the helium leak testing, the gas leakage (maximum leak rate: about 5×10⁻⁵ Pa·m³/s) was detected every time the helium gas was sprayed in the beryllium target of Example 1. Note that, the leak rate before the helium gas was sprayed was about 2×10⁻⁷ Pa·m³/s. Therefore, it was found that there were gaps enough to have helium permeability in the beryllium target of Example 1. The dynamic molecular size of helium is 0.26 nm, which is slightly smaller than the molecular size of hydrogen of 0.289 nm. Therefore, it is believed that when accelerated and emitted protons turn into hydrogen gas in the beryllium target of Example 1, the hydrogen gas is released outside of the beryllium target similarly with the helium gas, thus preventing generation of blistering. For the reference, FIG. 2 shows a graph plotting a leak rate relative to elapsed time in the helium leak testing using a beryllium target of Example 1.

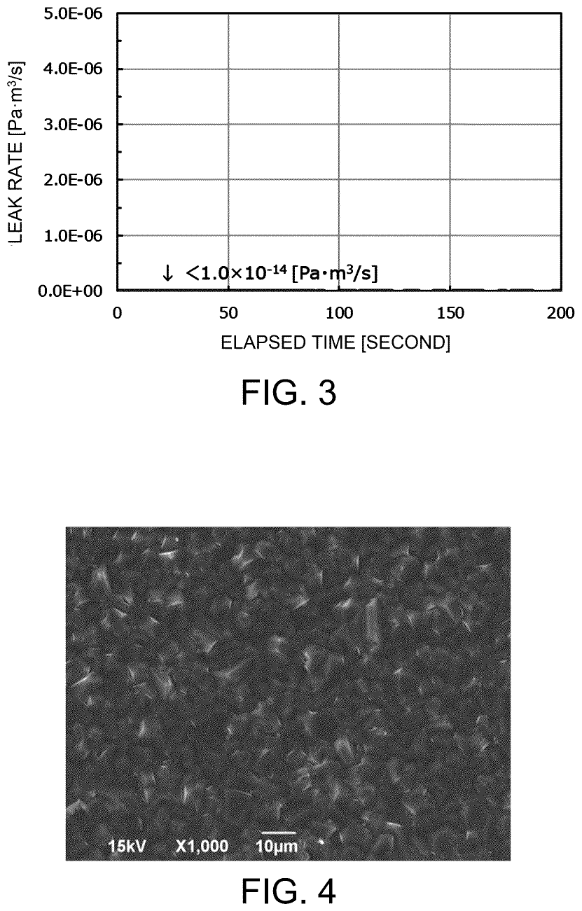

On the other hand, it was found that the beryllium target of Example 2 had a leak rate of less than a detection limit (1.0×10⁻¹⁴ Pa·m³/s) even when the helium gas was sprayed, and had no helium permeability. Therefore, it is believed that when accelerated and emitted protons turn into hydrogen gas in the beryllium target of Example 2, the hydrogen gas is not likely to be released outside the beryllium target, resulting in generation of blistering. For the reference, FIG. 3 shows a graph plotting a leak rate relative to elapsed time in the helium leak testing using a beryllium target of Example 2.

### <Tisssue Observation of Beryllium>

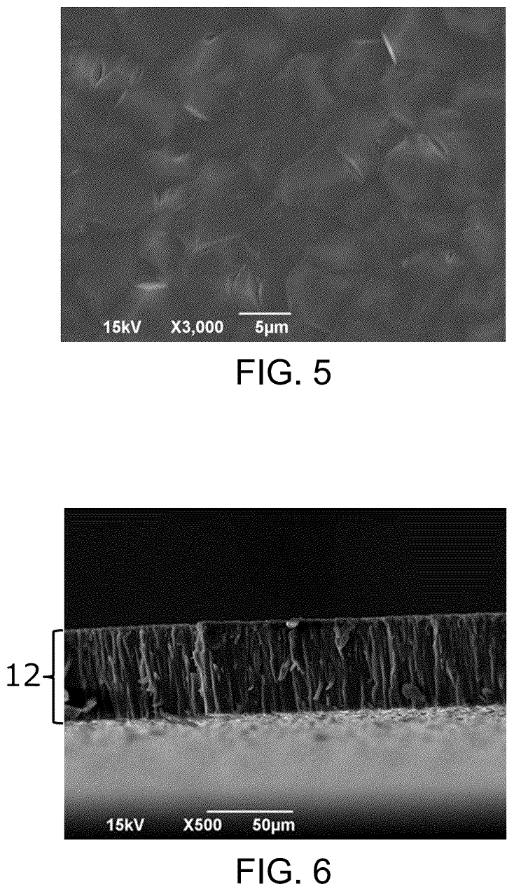

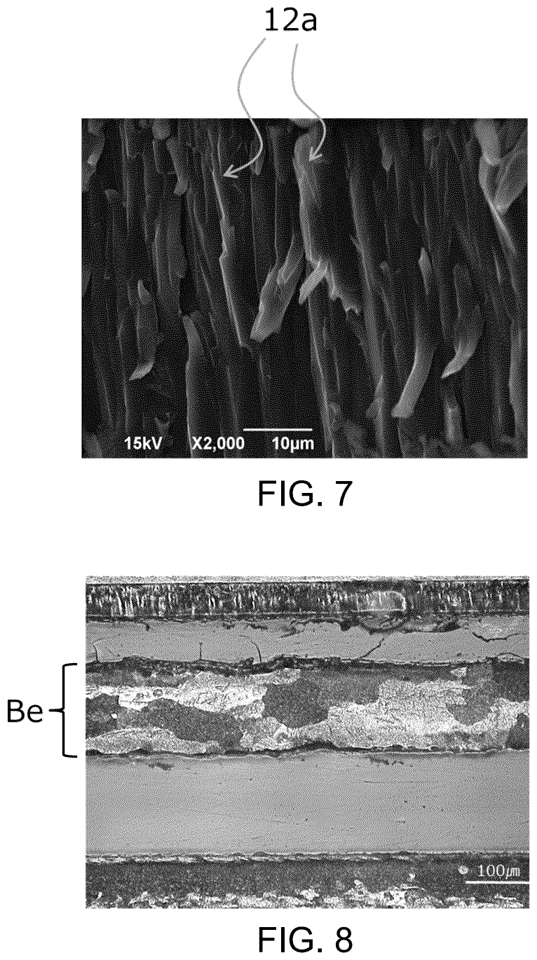

The beryllium target of Example 1 was observed from just above using a scanning electron microscope (SEM, available from JEOL Ltd., product number: JSM-6490LA) under the conditions of acceleration voltage of 15 kV and magnification of 1,000 folds and 3,000 folds. The obtained observation images were shown in FIG. 4 (magnification of 1,000 folds) and FIG. 5 (magnification of 3,000 folds). Then, a resin was embedded in the beryllium target of Example 1, followed by polishing the cross section. Then, the polished cross section of the beryllium target was observed using the above SEM under the conditions of acceleration voltage of 15 kV and magnification of 500 folds and 2,000 folds. The obtained observation images were shown in FIG. 6 (magnification of 500 folds) and FIG. 7 (magnification of 2,000 folds). As shown in FIGS. 4 to 7, it was confirmed that the beryllium target of Example 1 (beryllium layer 12) is composed of a plurality of beryllium crystal grains 12a having a columnar structure and has gaps between the plurality of beryllium crystal grains 12a.

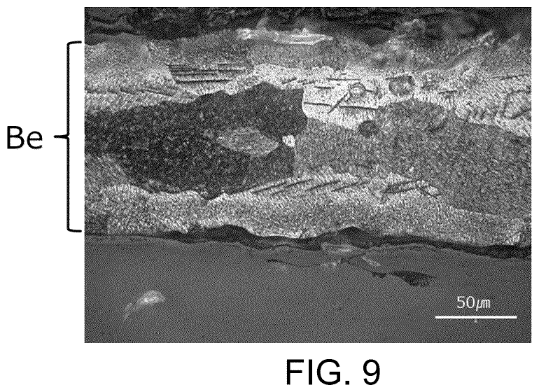

On the other hand, in the beryllium target of Example 2, it was difficult to confirm the metallic tissue in the same SEM observation as that of Example 1. Therefore, after an etching treatment, the cross section was observed. Specifically, a resin was embedded in the beryllium target of Example 2, followed by polishing the cross section. When an etching solution was brought into contact with the polished cross section to perform the etching treatment, the metallic tissue became obvious. The cross section obtained after the etching treatment was observed using an optical microscope at the magnification of 200 folds and 500 folds. The obtained observation images were shown in FIG. 8 (magnification of 200 folds) and FIG. 9 (magnification of 500 folds). As shown in FIGS. 8 and 9, it was confirmed that the beryllium target of Example 2 has flat crystal grains that exist closely to each other and had no gaps.

What is claimed is:

1. A neutron-generating target, comprising a beryllium layer composed of a plurality of beryllium crystal grains having a columnar structure that has grown in a thickness direction, wherein the beryllium layer has gaps between the plurality of beryllium crystal grains.

2. The neutron-generating target according to claim 1, wherein the plurality of beryllium crystal grains have a frost column-like structure as a whole.

3. The neutron-generating target according to claim 1, wherein a distance between centers of the plurality of beryllium crystal grains adjacent to each other is from 2 to 50 μm.

4. The neutron-generating target according to claim 1, wherein the beryllium crystal grains have an aspect ratio of from 1 to 3,000.

5. The neutron-generating target according to claim 1, wherein the beryllium layer has helium gas permeability.

6. The neutron-generating target according to claim 1, wherein the beryllium layer is a physical vapor deposition film.

7. The neutron-generating target according to claim 1, wherein the beryllium layer has a thickness of from 0.02 to 6 mm.

8. The neutron-generating target according to claim 1, further comprising a substrate on one surface of the beryllium layer.

9. The neutron-generating target according to claim 8, wherein the substrate is composed of copper.

10. A method for producing a neutron-generating target, the method comprising allowing a plurality of beryllium crystal grains to grow on one surface of a substrate in a thickness direction of the substrate by a physical vapor deposition, thereby obtaining a beryllium layer having gaps between the plurality of beryllium crystal grains.

11. The method for producing a neutron-generating target according to claim 10, wherein the physical vapor deposition is at least one selected from the group consisting of a resistance heating evaporation method, an electron-beam evaporation method, and a sputtering method.
